(19) United States
(12) Patent Application Publication
(10) Pub. No.: US 2009/0047218 A1
(43) Pub. Date: Feb. 19, 2009

(54) Method for producing albumin conjugates comprising an X-ray contrast medium as the active substance

(75) Inventors: Sinn Hansjorg, Wiesloch (DE); Mulbaler Marcel, Mauer (DE)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: Albupharm Heidelberg GmbH & Co. KG, Heidelberg (DE)

(21) Appl. No.: 11/919,683

(22) PCT Filed: May 11, 2006
(86) PCT No.: PCT/EP2006/004434

§ 371 Date: Oct. 31, 2007

(30) Foreign Application Priority Data
May 11, 2005 (DE) 10 2005 021 846.6

(51) Int. Cl. A61K 49/04; C07K 14/76; C07K 14/765

(52) U.S. Cl. 424/9.4; 530/362; 530/363

(57) ABSTRACT

The invention concerns X-ray contrast medium-protein conjugates and in particular X-ray contrast medium-albumin conjugates, medicaments comprising X-ray contrast medium-protein conjugates, processes for the production of such conjugates as well as the use thereof.

The present invention concerns X-ray contrast medium-protein conjugates and in particular X-ray contrast medium-albumin conjugates, medicaments containing X-ray contrast medium-protein conjugates, processes for producing such conjugates and the use thereof.

X-ray contrast media are substances which are introduced into the body to enhance differences in contrast in imaging techniques. In imaging diagnostics, contrast is understood as a difference in brightness within the image that is generated. Although almost all imaging techniques are suitable for showing body structures even without the aid of pharmaceutical preparations, contrast media are frequently used medically in order to enhance less pronounced differences in contrast between individual tissue types. Furthermore contrast media are used as markers of physiological processes such as for example blood flow, glomerular filtration and tubular secretion. They are either directly introduced into cavities or reach the target site (for example organs) by means of transport mechanisms. The mode of operation of contrast media is based on the fact that they increase (positive contrast media for example barium and iodine compounds) or lower (negative contrast media for example CO₂ gas, air, noble gases) the density of the radiated organ due to radiation absorption.

US 2005/0036946 describes radio-opaque polymeric compounds comprising at least one biodegradable and at least one iodinated radio-opaque end-group wherein the biodegradable region and the iodinated radio-opaque end-group are linked by at least one biodegradable bond.

DE 692 28 999 discloses polyaminated macromolecular compounds of biological or synthetic origin which are characterized in that they carry at least three iodinated radio-opaque derivatives as well as processes for their preparation and their use as contrast media.

Previously various triiodobenzoic acids in a low-molecular form have been used as contrast media such as e.g. diatrizoic acid, ioxitalamic acid, ioxaglic acid, iotroxic acid, iohexyl, iopentol and iodixanol. A disadvantage of the previously used substances is in particular the short residence time in the circulation such that they only have a very narrow time window for contrasting and are not suitable for directly imaging solid tumours by an enhanced contrast. Another disadvantage of the previously used X-ray contrast media is that only small amounts are taken up by living cells i.e. only a small proportion of the administered drug reaches the target site. Due to this low bioavailability of the X-ray contrast media they have to be administered in high doses which results in an unacceptably high number of undesired side-effects for a diagnostic agent. Observed side-effects are for example complaints and/or problems in the area of the injection site, of the entire cardiovascular system, of the kidney and of the central nervous system. Especially in the case of iodine-containing contrast agents these side-effects differ depending on the indication and extend from a sensation of heat and pain at the site of injection such as rubefacient, urtication, and also nausea, vomiting, heat sensation, tickling of the throat, inter alia to very severe reactions such as bronchospasm, asthma attacks, severe cardiovascular reactions such as circulatory collapse or tonic-chronic spasms which may lead to death. In the case of the ionic contrast agents the cause of these massive side-effects is due to the up to 8-fold higher osmolality of the contrast agents compared to normal physiological values. The administration of such strongly hypertonic preparations results in massive disturbances of the electrolyte balance. These are manifested among others as hypervolaemia, diuresis, vasodilation and a fall in blood pressure. In addition contrast media are able to trigger allergic and pseudoallergic reactions. Hence, corticoids are often administered prophylactically to reduce the risk in radiographic examinations. However, the corticoids also have a large spectrum of undesired side-effects such as acute/latent adrenal insufficiency, Cushing's syndrome, diabetes mellitus, increased risk of infection, disorders of wound healing, atrophy of the subcutaneous tissue, duodenal or gastric ulcers, myopathy, osteoporoses and psychic disorders.

One object of the present invention was therefore to provide X-ray contrast media in a form that enables the difficulties occurring in the state of the art to be overcome and in particular enables pathologically altered tissue sites to be visualized radiographically using a substantially lower dose of X-ray contrast medium with at the same time a longer half-life and which thus cause less severe side-effects in the organism.

This object is achieved according to the invention by providing an X-ray contrast medium-protein conjugate comprising a carboxyl group-containing X-ray contrast medium and a protein. Low-molecular active substances which would be rapidly eliminated from the body are hidden from the excretion and elimination mechanisms of the body by coupling X-ray contrast media to proteins and in particular to carrier proteins, and a long half-life and thus a high bioavailability in the body is achieved.

Successfully reacting X-ray contrast media with albumin was surprising in that the X-ray contrast media that are used are considered to be extremely unreactive and a person skilled in the art would have expected that their reactions would correspondingly also proceed incompletely even after a long activation period. Another surprising action of these X-ray contrast media was their long residence time after enzymatic cleavage of the protein in the cell.

The major advantage of using a protein conjugate compared to the low-molecular contrast medium is that the macromolecular contrast medium is now only released by enzymatic cleavage of the protein in the region of the pathologically altered tissue sites and in particular in the region of the proliferating tumour tissue and accumulates in these regions. The concentration is too low for a reliable contrasting in all other types of tissue. Since on the one hand, the administered dose is substantially lower and healthy cells do not take up or degrade albumin or its native conjugates in vivo, the previously common side-effects are greatly reduced or no longer occur at all. Another advantage of the protein conjugate as a contrast medium is that due to their accumulation in solid tumours, the previously very narrow time window for contrasting is substantially broadened without side-effects occurring. Furthermore the specific coupling of the contrast medium to a protein and in particular to albumin according to the invention, allows the osmolality of the contrast medium to be adjusted to a tolerable level for the organism.

The X-ray contrast medium is preferably directly covalently coupled to the protein. The direct coupling of X-ray contrast media to proteins and in particular to carrier proteins enables a specific production of stable, hydrolysis-insensitive X-ray contrast medium-protein conjugates without changing the physical properties of the X-ray contrast medium, without loss of the natural properties of the protein serving as the carrier and without forming ammonia. As a result the shelf life of these conjugates can be considerably extended. Hence the conjugates according to the invention have a particularly long storage life and shelf life. The shelf life is understood as the time at which the content of the drug or in the case of mixtures the content of the most labile component is decreased by 10% of the declared amount. If this time is less than three years, an expiry date must be provided on the package which gives the time at which 90% of the drug still remains in an unchanged form. The conjugate according to the invention preferably has a shelf life of ≧9 years, particularly preferably of ≧4 years and especially preferably of ≧7 years and most preferably of ≧10 years. Compared to previous common X-ray contrast medium-protein conjugates which comprise a linker such as for example cyanuric chloride, they have a substantially longer shelf life and thus also a greater drug safety.

A direct covalent coupling of the X-ray contrast medium to the carrier means that the X-ray contrast medium is bound to the transport protein by a linker-free or spacer-free bond. The X-ray contrast medium is preferably bound covalently to the protein by an acid amide bond which is formed from a carboxyl group of the X-ray contrast medium and an amino group, preferably a lysine group of the protein.

Alternatively the X-ray contrast medium and a protein can be reacted with a linker in which case the reaction is preferably by means of a linkage by covalent bonds.

In a preferred embodiment of the present invention the X-ray contrast medium is covalently bound to the protein by an acid amide bond which is formed from a carboxyl group of the X-ray contrast medium and an amino group of the protein.

The covalent coupling preferably takes place by means of a carbodiimide as an activation reagent where the carbodiimide is particularly preferably N-(3-dimethylaminopropyl)-N′-ethylcarbodiimide. In this case a conjugate is especially preferred which can be obtained by reacting an X-ray contrast medium and a protein in the presence of a carbodiimide as an activation reagent without N-hydroxysuccinimide and/or without N-hydroxysuccinamide. An X-ray contrast medium and a protein are particularly preferably reacted in the presence of a carbodiimide as the activation reagent without additional activation reagent. Alternatively the conjugate can be obtained by firstly forming a succinimidyl ester from the X-ray contrast medium using a carbodiimide and N-hydroxysuccinimide and subsequently reacting the succinimidyl ester of the X-ray contrast medium with the protein.

In another preferred embodiment the conjugate can be obtained by using thionyl chloride or oxalyl chloride to produce an acid chloride of the X-ray contrast medium from the carboxyl-group-containing X-ray contrast medium and subsequently directly reacting the acid chloride with the protein.

The use of iodine compounds and in particular triiodobenzoic acid as X-ray contrast medium has proven to be particularly suitable.

In a preferred embodiment of the present invention suitable triiodobenzoic acids are selected from the group comprising 2,3,5-triiodobenzoic acid, diatrizoic acid, ioxitalamic acid, ioxaglic acid and/or iotroxic acid.

According to the present invention the commercially available 2,3,5-triiodobenzoic acid is particularly preferably bound to a carrier protein.

Albumin and in particular serum albumin and most preferably human albumin or human serum albumin (HSA) is preferably used as a protein in the conjugates according to the invention. Human albumin is an endogenous ubiquitously distributed and non-immunogenic protein.

It has a molecular weight of about 68 kDa and is thus not eliminated by the kidneys. Albumin constitutes approximately 60% of the total amount of plasma protein. In the healthy organism it fulfils among others transport functions for many substances and in an acute emergency it serves as a reserve energy carrier which is available everywhere and at anytime in the organism.

It is not taken up by healthy cells under physiological conditions. In contrast, cells associated with inflammatory processes and cells associated with tumours and in particular with solid tumours have a high turnover of proteins and in particular of plasma proteins and mainly of albumin. This means that by coupling X-ray contrast media according to the invention to proteins and in particular albumin, it is possible to achieve a targeted accumulation of the contrast medium at the site of action and in particular in solid tumours and other pathologically altered tissue sites. This targeted accumulation allows a substantial reduction of the dosage of active substance which facilitates a treatment with few side-effects because the active substances are now only released in the area of pathologically altered tissue sites. Another advantage of albumin is that it is available even in large amounts anytime in a clinically usable form.

The biokinetic behaviour and thus also the biological half-life of the conjugate according to the invention is determined solely by the macromolecule albumin but not by the low-molecular X-ray contrast medium. The protein used according to the invention to form the conjugates preferably has a molecular weight of ≧18,000 Da, particularly preferably of ≧30,000 Da and especially preferably of ≧50,000 Da.

The X-ray contrast medium is preferably coupled to the carrier protein which is preferably albumin without limiting the biological efficacy of the active substance and without loss of the natural character of the protein used as the carrier and in particular of the albumin.

A protein which is present in its natural form is understood especially as a non-denatured, non-altered protein and in particular a protein whose properties such as for example its structure, its physiological properties etc. are unchanged. The conjugates of the invention preferably contain an X-ray contrast medium and a protein in a molar ratio of 2:1 to 0.1:1, preferably of 1.1:1 to 0.5:1 and particularly preferably of 1.1:1 to 0.9:1. In particular a molar ratio of about 1:1 is advantageous. Thus for example albumin still exhibits a biologically active behaviour at a 1:1 loading with an X-ray contrast medium. This advantageously ensures that albumin as a carrier of the X-ray contrast medium still has a distribution space in the body which is identical to that of natural HSA even after the loading. Consequently it is unnecessary to know the position of the tumour and hence the conjugate is administered systemically so that the tumour can subsequently be visualized positively. Thus in order to visualize the cells that are affected by the tumour, it is not necessary to inject the conjugate directly into the tumour or into its immediate vicinity. Another advantage of the natural character of the albumin is that the staining of the tumour cells is due on the one hand, to the natural distribution pattern of albumin in the extravascular space and, on the other hand, to the high uptake of albumin by tumour cells.

With regard to the X-ray contrast medium the coupling preferably takes place without changing the physical properties of the contrast medium.

In principle it is preferable to use a protein that is native to the patient for which the conjugate is intended. This means that the protein is present in a native form and furthermore that for example human proteins are used for administration to humans and corresponding mouse proteins are used for administration to mice. Hence a native protein is a protein which originates from the same species as the species to which the protein is administered.

The coupling of the X-ray contrast medium to the carrier protein preferably albumin, preferably takes place without limiting its native character. The active substance is particularly preferably covalently coupled to the carrier protein. Furthermore, the covalent coupling is preferably selected such that it can be cleaved again under physiological conditions for example in healthy and pathologically altered tissues such that the biological efficacy of the original active substance is retained and can be utilized. The cleavage preferably occurs enzymatically. It can either be bound directly to the protein or via a linker.

Thus, according to the invention X-ray contrast medium-protein conjugates and in particular X-ray contrast medium-albumin conjugates are formed without changing the biological efficacy of the active substance and without loss of the native character of the protein used as a carrier and in particular of the albumin.

Furthermore, within the scope of the present invention low-molecular X-ray contrast media are particularly preferably bound to the transport protein because in this manner it is solely the macromolecule albumin which determines the biokinetic properties but not the low-molecular X-ray contrast medium. The X-ray contrast medium used according to the invention to form the conjugates preferably has a molecular weight of <2,000 Da, particularly preferably of <1,000 Da and especially preferably of <500 Da.

In a preferred embodiment the conjugate according to the invention is an active ingredient in a diagnostic or therapeutic agent. Such a pharmaceutical preparation has in particular low side-effects and can for example also be administered to outpatients. It is preferably administered intravenously.

One dosage unit preferably contains 1 to 2 mg active substance, X-ray contrast medium per kilogram body weight and in particular 0.9 to 1.5 mg active substance per kilogram body weight. The dose can in particular be chosen to be lower than that used for conventional therapy with X-ray contrast medium and is preferably ≦9 mg and particularly preferably ≦0.5 mg X-ray contrast medium per kilogram body weight.

In a preferred embodiment of the present invention the X-ray contrast medium-protein conjugate is used to produce a medicament for visualizing tumours. It is particularly preferably used to positively visualize solid tumours. In particular the medicament is a contrast medium in X-ray diagnostics.

Another important aspect of the invention concerns the use of the conjugate described herein and in particular as defined in one of the claims 7 to 23 to produce a medicament for treating tumours. The inventors have discovered for the first time that X-ray contrast media cannot only be used as diagnostic agents but also as therapeutic agents in particular as radiation sensitizers that can be accumulated in the tumour.

The mode of action of the contrast agents is based on the fact that they increase the density of the radiated organ by radiation absorption. It has been known for a long time that X-ray contrast media, and in particular X-ray contrast media containing iodine can trigger side-effects that can occur during or after the examination i.e. after administering the X-ray contrast medium and can be attributed to different causes. A distinction must be made between acute side-effects directly during the administration and long-term side-effects a few days after the examination.

The former side-effects include heat sensation and pain at the site of injection, vasodilation associated with a fall in blood pressure and attacks of dizziness, diuresis etc. This is due to the unphysiologically high osmolarity values of the administered contrast media solutions which can be 1400 mOsm (milliosmoles) and more whereas the physiological value for osmolarity is only about 284 mOsm. Such high osmolarities cause massive disturbances in the water-electrolyte balance especially in the direct vicinity of the site of application but not only there.

These acute side-effects can be avoided by chemically binding low-molecular compounds to endogenous macromolecules such as for example albumin in a suitable manner. The reason for the use of albumin as a carrier substance is due to the sum of the properties of albumin listed above and in particular its high specificity and selectivity for tumour cells as well as the long half-life of albumin. In the prior art X-ray contrast media have previously only been used to visualize blood vessels and not for the positive visualization of tumours. One subject matter of the present invention is the use of the X-ray contrast medium-albumin conjugates according to the invention for the positive visualization of tumours. Substantially higher amounts of contrast medium would usually be required for this which can be advantageously dispensed with according to the invention due to the binding of the contrast medium to albumin which has a very long half-life. Thus it is possible to positively visualize tumours with the same or even a lower amount of administered contrast medium. This substantial reduction in the required amounts of contrast medium is an important factor for reducing the risk of undesired side-effects.

The long-term side-effects especially of iodine-containing X-ray contrast media which occur a few days after the examination include metabolic disorders in patients who have a tendency for thyroid hyperfunction or have an existing hyperfunction of the thyroid gland. In such cases increased cardiac palpitation, high blood pressure, increased unrest with sleeplessness and sweats and diarrhoea occur most frequently. The cause of these undesired late side-effects is iodine which is released during the examination. Iodide is in turn formed when elemental iodine which is released into the circulation when energy-rich X-rays are absorbed by the contrast medium, reacts with further reaction partners such as for example tyrosine residues of albumin. This iodide which is formed at the same time when elemental iodine reacts with tyrosine residues is ultimately the trigger for hyperthyroidism.

The present inventors have now for the first time discovered that these late side-effects which occur a few days after the examination not only represent hyperthyroidism due to released iodide but also a destruction of cells due to the intracellular release of elemental iodine. The prior art have previously assumed that X-ray contrast media only have a physical action but no pharmacological action whatsoever. The conventional low-molecular, non-tumour-accessible X-ray contrast media of the prior art were used exclusively to absorb radiation to produce radiographs for diagnostic purposes. However, attention was never paid to the iodide released by the radiation absorption although the time-delayed side-effects are due to this effect of iodine released by X-rays.

The present inventors have for the first time also discovered a pharmacological action of X-ray contrast media in particular of iodine-containing X-ray contrast media i.e. the destruction of tumour cells by means of these X-ray contrast media. A targeted and specific destruction of tumour cells can, however, only be achieved by coupling the X-ray contrast medium to a protein and in particular albumin.

Hence, a further aspect of the present invention concerns the use of an X-ray contrast medium-protein conjugate according to the invention to produce a medicament for treating tumours. The tumours are preferably treated by means of the fact that toxic entities are released when the X-ray contrast medium is irradiated which are preferably released intracellularly and into the circulation when energy-rich X-rays are absorbed by the contrast medium. Within the scope of the present invention the toxic entities are preferably radicals and in particular halogen radicals where among the halogen radicals which can have iodine, bromine and/or fluorine as the halogen, above all iodine radicals are especially preferred. In addition the toxic entities can for example be benzoic acid radicals.

An energy source having an energy of >10 electron volts (eV), more preferably of >100 eV and most preferably of >1 kiloelectron volts (keV) is preferably used according to the invention for the irradiation. In a particularly preferred embodiment the irradiation takes place using an energy source which radiates energy in a range of 20 to 25 keV. The radiation dose that is used can, according to the invention, be in the range of the radiation dose used for diagnostics or below this range in order to keep the radiation burden to the body as low as possible. Alternatively it is also possible to use a higher radiation dose than is necessary for diagnostics.

The mechanism of action of the X-ray contrast medium-protein conjugate is in particular as follows: After administering the conjugate, it is transported with high specificity and selectivity into the tumour tissue, on the one hand, due to the natural distribution pattern of albumin in the extravascular space and, on the other hand, due to the high albumin uptake of the tumour cells, where albumin is then converted by the tumour cells. When the X-ray contrast medium that is now present intracellularly is irradiated, the energy-rich radiation that is beamed in which has a higher energy than the binding energy between the radicals to be generated in the X-ray contrast medium results in the formation of intracellular toxic entities, preferably radicals, particularly preferably halogen radicals and in particular iodine radicals as well as for example benzoic acid which is only poorly soluble at a physiological pH. These intracellular toxic entities can have fatal consequences when using a conventional unspecific X-ray contrast medium. The binding energy between the iodine and the benzene ring is for example about 5 to 6 eV, whereas the energy of the radiation released by the radiation source is much higher and approximately 1000-fold higher, i.e. 20 to 25 keV. Hence in a preferred embodiment of the invention the energy of the radiation that is used to irradiate the conjugate according to the invention is 10-fold, preferably 100-fold and most preferably 1000-fold higher than the binding energy of the bond in the X-ray contrast medium that is to be cleaved into radicals. However, according to the invention the toxic entities accumulate specifically in the tumour cells due to the coupling of the X-ray contrast medium to albumin and destroy the tumour cells containing the contrast medium in a targeted manner. Example 4 shows that iodine-containing X-ray contrast media are not stable towards energy-rich radiation. In particular the X-ray contrast medium has been demonstrated to be unstable towards UV radiation which is even about 100-times less than X-ray radiation.

In a preferred embodiment of the present invention the protein-X-ray contrast medium conjugate can be used effectively for the targeted destruction of tumour cells without requiring an increase of the amount of conjugate or of the radiation dose. The radiation dose can preferably also be increased.

A further aspect of the present invention concerns the use of the conjugate according to the invention to produce a medicament to protect tissue which is unaffected by tumours from radicals. Because healthy cells do not consume albumin under physiological conditions, the albumin present in the conjugate according to the invention serves to protect tissue(s) that has/have not been affected by tumour(s) and in particular healthy tissue(s) and body fluids in which the administered conjugate according to the invention is present unspecifically which is for example tissue of the transport path to the tumour cells, tissue surrounding the tumour tissue as well as its blood circulation, from radical stress during the irradiation. The tyrosine residues in the albumin present in healthy tissue or in the blood circulation act as radical scavengers for the iodine radicals of the X-ray contrast medium that are formed during the irradiation.

Thus albumin has a total of two effects i.e., on the one hand, an extended half-life of albumin in the body and a targeted uptake into tumour cells and, on the other hand, a protection against radicals that are formed during the irradiation in tissue that is not affected by tumours but is still affected by the radiation.

Hence a further embodiment of the invention is a composition which comprises an X-ray contrast medium-protein conjugate and at least one radical scavenger which further supports the anti-radidal effect of the protein and in particular of the albumin. Surprisingly this enables synergistic effects to be achieved with regard to protecting tissue that has not been affected by the tumour from radicals. In this connection the radical scavenger is present in the composition in an unbound, non-conjugated form.

Radical scavengers are understood as organic or inorganic substances which react chemically with reactive radicals to form more stable compounds. The addition of radical scavengers to a radical chain reaction thus also interrupts the reaction chain. In this case radical scavengers are particularly preferred according to the invention which are pharmaceutically and physiologically acceptable. These include L-cysteine, cysteamine, melamine, glutathione, uric acid and liponic acid and dihydroliponic acid which, as radical scavengers, act in the tissue to protect it against damaging influences. Radical scavengers which are used for cancer prophylaxis are particularly preferred. These include vitamin A, retinoids and β-carotene (provitamin A) and the antioxidative vitamins C and E. As radical scavengers they protect the DNA and cell membranes from oxidative damage by radicals and thus contribute to the protection from mutations and to the maintenance of cell integrity. They also include. Vitamin E and albumin are particularly preferred within the scope of the present invention as radical scavengers, albumin being especially preferred because it is an efficient iodine radical scavenger due to its tyrosine residues.

A further aspect of the present invention is a diagnostic or therapeutic agent which comprises an X-ray contrast agent-protein conjugate in combination with at least one radical scavenger as the active substance.

In another preferred embodiment of the present invention the conjugate according to the invention as well as the composition comprising this conjugate and at least one radical scavenger are therefore used to produce a medicament for protecting tissue that is not afflicted by tumours from radicals.

Yet a further aspect of the present invention concerns the use of the conjugate according to the invention for therapeutic monitoring after treatment with the conjugate and in particular three to four weeks after the treatment. The long biological half-life of albumin as well as of the iodine which is released during irradiation of the X-ray contrast medium allows a direct therapeutic monitoring preferably without addition of additional X-ray contrast medium-albumin conjugate which would be an additional burden for the body even after a time span of three to four weeks after administration of the conjugate. Thus therapeutic monitoring with low side-effects is possible at a later time so that even if the procedure is repeated several times the radiation dose is still within the range of diagnostic measures.

Thus the X-ray contrast medium-protein conjugate and in particular the 2,3,5-triiodobenzoic acid-albumin conjugate causes neither side-effects directly during the administration nor long-term side-effects several days after the examination.

A further subject matter of the present invention is a process for producing an X-ray contrast medium-protein conjugate comprising reacting a carboxyl group-containing X-ray contrast medium preferably a low-molecular carboxyl group-containing X-ray contrast medium with a protein preferably a high-molecular carrier protein. The X-ray contrast medium is preferably directly covalently coupled to the protein for example albumin without limiting its native and natural character. A coupling has proven to be particularly advantageous in which firstly an activated acid is formed from the low-molecular contrast medium by means of a carbodiimide and subsequently the activated acid of the X-ray contrast medium is reacted with a protein.

For the production of the conjugates that are used according to the invention it is important that the active substance is efficiently coupled to the carrier molecule (i.e. the protein). In particular undesired alterations of the carrier protein or/and of the active substance should not occur during the coupling. Conventional activation of organic compounds containing carboxyl groups with dicyclohexylcarbodiimide (DCC) requires more than 12 hours at room temperature or at +4° C. (P. Hammer and W. Heeschen, “Milchwissenschaft” 1995, 50(9), pages 513-514, DP 41 22 210 A1; EP 0 879 604 A1; EP 0 820 308). Moreover, in this process insoluble substances are formed during the activation some of which already precipitate during the activation and when the activated active substance is fed into an aqueous protein solution and have to be separated by time-consuming and expensive filtration steps that are never 100% (due to the lipophilic domains in albumin) in addition to the actual product purification in order that the conjugate can be administered for medical purposes. Therefore it is preferred according to the invention that the carbodiimide is N-(3-dimethylaminopropyl)-N′-ethylcarbodiimide hydrochloride.

It was surprisingly found that by using EDC especially in the form of its hydrochloride it is possible to activate the carboxyl group-containing organic compound and react it with a carrier protein without forming water-insoluble by-products which would have to be separated in a time-consuming and costly manner. In this process intermediate purification steps are unnecessary and the preparation time and thus also the production costs are substantially reduced. Furthermore problems that can be caused by insoluble substances or by-products when the conjugate is injected into a human or animal body are avoided.

The activation preferably takes place at a temperature of 10 to 100° C., more preferably of 20 to 90° C. and still more preferably of 50 to 75° C. for a reaction period of 1 to 10 hours, more preferably of 20 to 50 minutes. The activated active substance is preferably reacted with the carrier protein at a temperature between 10 and 50° C., in particular between 20 and 40° C.

The carboxyl group-containing compound, in particular 2,3,5-triiodobenzoic acid is preferably activated with EDC in an organic solvent preferably in dimethyl sulfoxide (DSMO). Further suitable organic solvents are for example dimethyl-acetamide or dioxane. The activation is preferably carried out with the exclusion of water in particular in the presence of ≦5% by weight water, more preferably of ≦1% by weight water and most preferably completely anhydrously.

In an alternative embodiment the production process is carried out using the activation reagents EDC and N-hydroxysuccinimide. An advantage of these activation reagents is that they are highly soluble in water. As a result coupling reagents that are not consumed during the reaction can be simply removed from the product obtained for example by washing with water. In contrast when the prior art coupling reagents are used for example when using dicyclohexylcarbodiimide (DCC) an inseparable residue of coupling reagent remains in the conjugate. When using DCC for a X-ray contrast medium-albumin conjugate an inseparable residue of about 13 to 15% by weight DCC is observed in the conjugate which is probably bound to a lipophilic domain in albumin. This residue can only be detected with the aid of HPLC and can only be preparatively separated in a very time-consuming manner.

If in addition a high excess of DCC relative to the active substance to be coupled is used as described in the publication by P. Hammer and W. Heeschen (see above), i.e. a molar ratio of DCC:active substance of about 10:1, it is no longer possible to completely separate the protein by means of dialysis or ultrafiltration. However, this DCC that adheres to the protein and which is still reactive results in a progressive alteration of the protein in the course of time due to intramolecular and intermolecular cross-linking resulting in a time-dependent change in the properties of the carrier protein. Thus a conjugate produced in this manner is not suitable for clinical use.

Hence a further preferred aspect of the invention concerns a process for producing a conjugate according to the invention comprising reacting an X-ray contrast medium with albumin in which an X-ray contrast medium and albumin are reacted in the presence of a carbodiimide preferably in the presence of N-(3-dimethylamino-propyl)-N′-ethylcarbodiimide without H-hydroxysuccinimide or N-hydroxysuccinamide or without any additional activation reagent.

It was surprisingly found that the optimized process which does not use N-hydroxysuccinimide or N-hydroxysuccinamide or other additional activation reagents has a positive effect on the purification procedure which simplifies the production. Due to the use of EDC alone for the activation without the addition of N-hydroxysuccimmide (HSI) or N-hydroxysuccinamide (HSA), the activation time of the X-ray contrast medium is substantially shorter than the 30 minutes required when using HSI or HSA. Another advantage of the optimized process is that after adding the activated active substance to the protein and in particular albumin without N-hydroxysuccinimide it is possible to directly monitor the coupling efficiency. When using N-hydroxysuccinimide it also has a high UV absorption in the HPLC when the UV measuring cell is adjusted to 280 nm and interferes or makes it more difficult to directly determine the coupling yield due to its retention time of 11.5 minutes at which other low-molecular compounds also appear. This means that in many cases the yield can only be determined at the end of the purification of the conjugate. This factor can now be excluded by the optimized process which does not use N-hydroxysuccinimide or N-hydroxysuccinamide. This is also a major advantage for product safety. Another advantage of the optimized process is that the coupling yield is surprisingly 98 to 99% on average. Thus the total costs of the respective conjugate are considerably reduced by this simplification of the production.

According to a further preferred coupling according to the invention an acid chloride of the X-ray contrast medium is produced from the low-molecular X-ray contrast medium using thionyl chloride or oxalyl chloride and the acid chloride is subsequently directly directed with the protein. The reaction of the X-ray contrast medium, preferably 2,3,5-triiodobenzoic acid, with thionyl chloride or oxalyl chloride is preferably carried out at a temperature of 10 to 100° C., more preferably of 20 to 90° C. and even more preferably of 50 to 75° C. for a reaction time of 1 minute to 10 hours, more preferably of 2 minutes to 50 minutes and most preferably of 5 to 15 minutes.

The molar ratio of the X-ray contrast medium to protein is preferably 21:1 to 0.1:1 and the protein is preferably albumin in the process for producing the conjugate according to the invention.

The invention is further elucidated by the following examples and the attached figures.

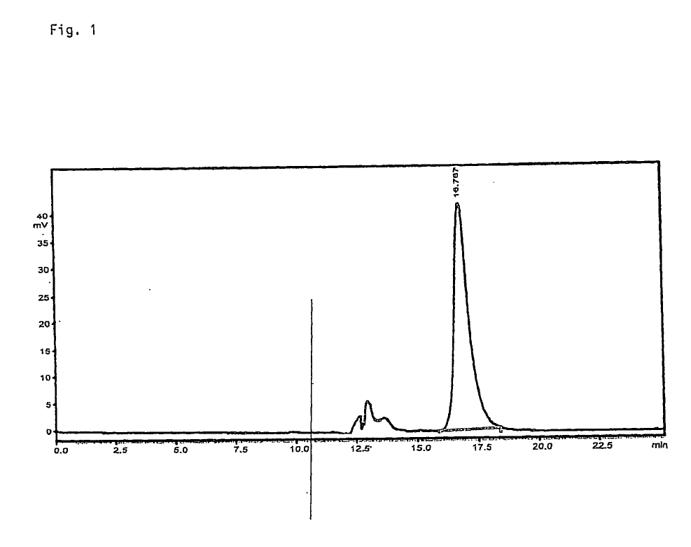

FIG. 1 shows a HPLC chromatogram of 2,3,5-triiodobenzoic acid alone.

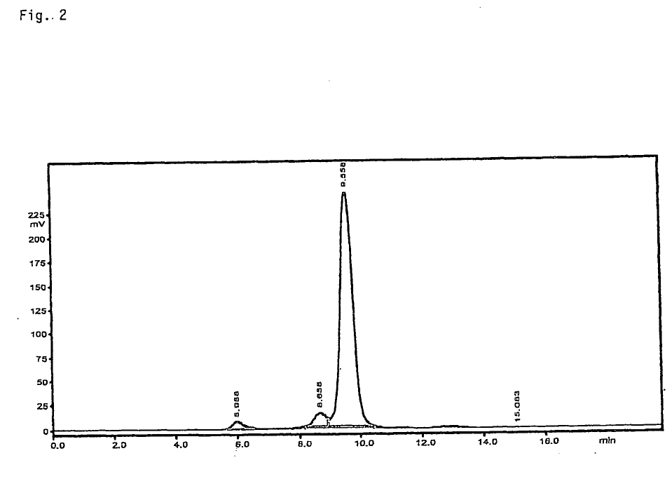

FIG. 2 shows the HPLC chromatogram of the 2,3,5-triiodobenzoic acid-HSA conjugate prepared according to example 1 (end product after purification.

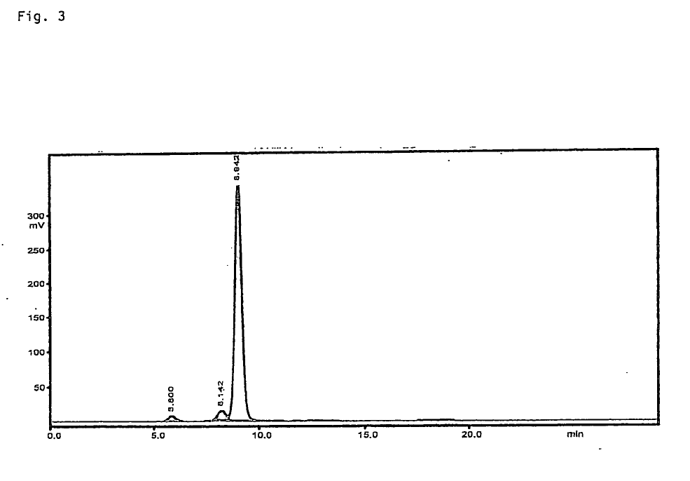

FIG. 3 shows the HPLC chromatogram of the 2,3,5-triiodobenzoic acid-HSA conjugate prepared according to example 2 directly after the production and before the purification.

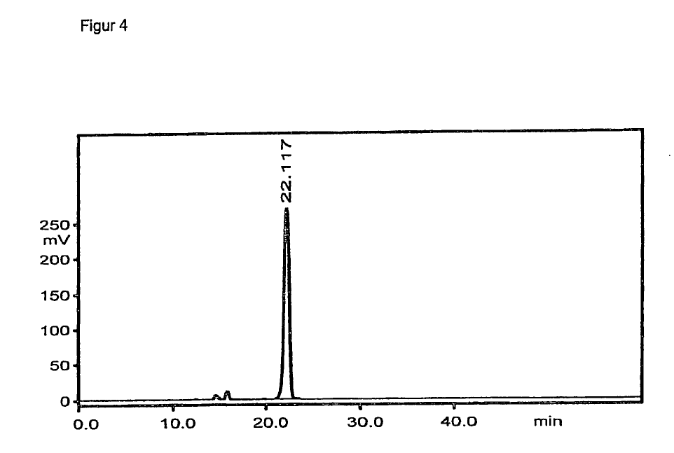

FIG. 4 shows the HPLC chromatogram of 2,3,5-triiodobenzoic acid alone.

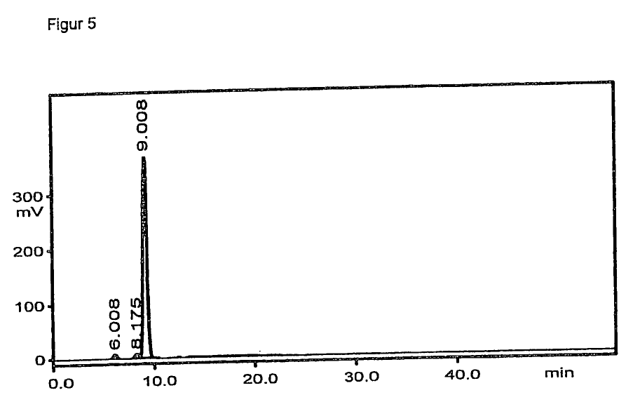

FIG. 5 shows the HPLC chromatogram of the 2,3,5-triiodobenzoic acid-HSA conjugate prepared according to example 3.

### STARTING MATERIALS

2,3,5-Triiodobenzoic acid (SIGMA-ALDRICH, Tauflcirchen), thionyl chloride (SIGMA-ALDRICH, Taufkirchen) and N-(3-dimethylaminopropyl)-N′-ethylcarbodiimide hydrochloride (SIGMA-ALDRICH, Taufkirchen) and albumin (Göricke, Dessau).

Other substitution patterns can also be used instead of the 2,3,5-substitution of the benzoic acid.

### Standard Preparation on a Laboratory Scale:

### EXAMPLE 1

Approximately 21.7 mg 2,3,5-triiodobenzoic acid (MW 499.8) are placed together with about 12.6 mg N-(3-dimethylaminopropyl)-N′-ethylcarbodiimide hydrochloride (MW 191.7; molar ratio 1:1.5) in a test tube with an NS 14.5 ground glass joint and stopper. After adding 1 ml dimethyl sulfoxide (DMSO) the reaction mixture is placed in a water bath preheated to 65° C. After a reaction time of about 30 minutes, a clear colourless solution of the activated triiodobenzoic acid is present which, after cooling to room temperature, is very slowly fed into a 5% albumin solution. A turbidity is briefly formed at the inlet site which, however, rapidly redissolves. The control chromatogram can be prepared directly after the coupling. The yield of the reaction is 99%.

The undesired accompanying substances: DMSO, N-(3-dimethylaminopropyl)-N′-ethyl-urea and non-covalently bound triiodobenzoic acid in the end-product are separated by ultrafiltration (YM 30, Millipore).

### EXAMPLE 2

Approximately 100 mg 2,3,5-triiodobenzoic acid (MW 499.8) is added to 2 ml thionyl chloride or oxalyl chloride and placed in a water bath preheated to 65° C. After about 10 minutes a pale yellow coloured clear solution of the desired acid chloride is present. Excess thionyl chloride or oxalyl chloride is completely removed in a vacuum. The weakly coloured solid residue is dissolved in 4 ml 1,4-dioxane and an 880 μl aliquot (corresponding to 22 mg 2,3,5-triiodobenzoic acid) is added directly to a 5% 0.17 M bicarbonate solution of albumin.

The quality control can be carried out immediately after the production.

### Quality Control (HPLC/SEC):

| | |
|---|---|
| Precolumn: | Reprosil 200 SEC 5 × 4 mm, 5 μm (Dr. Maisch GmbH) |
| Column: | Reprosil 200 SEC 300 × 4.6 mm, 5 μm (Dr. Maisch |
| | GmbH) |
| Mobile solvent: | 0.18 M Na₂HPO₄; pH 7.4; 5% methanol |
| Flow rate: | 0.3 mL/min |
| Pressure: | about 50 bar |
| UV-VIS | 280 nm |

### Retention Times:

| | | |
|---|---|---|
| oligomeric albumin fraction | 5.92 | min |
| dimeric albumin fraction | 8.63 | min |
| monomeric albumin fraction | 9.55 | min |
| free 2,3,5-triiodobenzoic acid | 16.88 | min |

### Chromatograms: see FIGS. 1 to 3

### EXAMPLE 3

### Standard Preparation on a Laboratory Scale

Approximately 22 mg 2,3,5-triiodobenzoic acid (MW 499.81, about 44 μmol) is placed together with 1 ml thionyl chloride in a test tube with an NS 14.5 ground glass joint and reflux cooler. The reaction mixture is heated for about 30 minutes to 80° C. and subsequently excess thionyl chloride is removed in a vacuum from the now clear solution. The solid, dry residue is dissolved in 2 ml 1,4 dioxane and this solution is slowly added to a 5% albumin solution buffered with bicarbonate. A white turbidity is briefly formed at the inlet site which, however, rapidly redissolves. The control chromatogram can be prepared directly after the coupling by means of thin-layer chromatography (TLC) or high performance liquid chromatography (HPLC) or size exclusion chromatography (SEC). The yield of the reaction is 295%.

The undesired accompanying substances dioxane and non-bound triiodobenzoic acid are separated by means of ultrafiltration (YM 30, Millipore).

### Quality Control (HIPLC/SEC):

| | |
|---|---|
| Precolumn: | Reprosil 200 SEC 10 × 4 mm, 5 μm (Dr. Maisch GmbH) |
| Column: | Reprosil 200 SEC 300 × 4.6 mm, 5 μm (Dr. Maisch |
| | GmbH) |
| Mobile solvent: | 0.13 M Na₂HPO₄; 7.5% methanol, pH 7.2; |
| Flow rate: | 0.3 mL/min |
| Pressure: | about 50 bar |
| UV-VIS | 254 nm |

### Retention Times:

| | | |
|---|---|---|
| oligomeric albumin fraction | 6.00 | min |
| dimeric albumin fraction | 8.17 | min |
| monomeric albumin fraction | 9.00 | min |
| free 2,3,5-triiodobenzoic acid | 22.12 | min |

### Chromatograms: see FIGS. 4 and 5

### EXAMPLE 4

A simple experimental arrangement can be used to prove that X-ray contrast media containing iodine are not stable against energy-rich radiation.

Approximately 40 mg 2,3,5-triiodobenzoic acid (TIB) or another X-ray contrast medium that can be obtained commercially (diatrizoic acid or iotalamic acid etc.) is dissolved in 40 ml of a 0.1% starch solution while gently heating. 20 ml is in each case distributed to two Petri dishes (diameter 11.5 cm). Dish 1 is placed in full sunlight for 1 hour. Dish 2 is irradiated at a distance of 11 cm with a UV hand-held lamp (254 nm, 14 μW/cm²).

While the solution in dish 1 is still colourless after being exposed to daylight, dish 2 shows the typical, intensive blue staining of the iodine-starch reaction. This is even more significant because the energy of the UV irradiation (like the intensity of the irradiation source used) is approximately 100-fold lower than that of the X-ray irradiation of a computer tomograph.

1. Use of an X-ray contrast medium-albumin conjugate, comprising a carboxyl group-containing X-ray contrast medium and albumin for the preparation of a medicament for the radiographic treatment, visualization of tumours or a combination of them.

2. The use according to claim 1,
characterized in that
toxic entities are released during irradiation of the X-ray contrast medium.

3. The use according to claim 2,
characterized in that
the toxic entities are radicals, preferably iodine radicals.

4. The use according to claim 2,
characterized in that
tumour cells are destroyed when the X-ray contrast medium is irradiated.

5. The use according to claim 1,
characterized in that
the medicament is a contrast medium in X-ray diagnostics.

6. The use according to claim 1 to positively visualize tumours.

7. An X-ray contrast medium-albumin conjugate, comprising a carboxyl group-containing X-ray contrast medium and albumin,
obtainable by
reacting a carboxyl group-containing X-ray contrast medium and albumin in the presence of N-(3-dimethylaminopropyl)-N′-ethylcarbodiimide as the activation reagent without N-hydroxysuccinimide and/or N-hydroxysuccinamide, wherein the activation of the X-ray contrast medium is carried out with the exclusion of water.

8. An X-ray contrast medium-albumin conjugate, comprising a carboxyl group-containing X-ray contrast medium and albumin,
obtainable by
producing an acid chloride of the X-ray contrast medium from the carboxyl group-containing X-ray contrast medium using thionyl chloride or oxalyl chloride and subsequently directly reacting said acid chloride with albumin.

9. The X-ray contrast medium-albumin conjugate according to claim 7,
obtainable by
directly covalently coupling the X-ray contrast medium to albumin.

10. The X-ray contrast medium-albumin conjugate according to claim 9,
obtainable by
coupling the X-ray contrast medium to albumin via an acid amide bond.

11. The X-ray contrast medium-albumin conjugate according to claim 7,
obtainable by
reacting a carboxyl group-containing X-ray contrast medium and albumin in the presence of N-(3-dimethylaminopropyl)-N′-ethylcarbodiimide as the activation reagent without an additional activation reagent.

12. The X-ray contrast medium-albumin conjugate according to claim 7,
characterized in that
the X-ray contrast medium is an iodine compound.

13. The X-ray contrast medium-albumin conjugate according to claim 7,
characterized in that
the X-ray contrast medium is a triiodobenzoic acid.

14. The X-ray contrast medium-albumin conjugate according to claim 13,
characterized in that
the triiodobenzoic acid is a 2,3,5-triiodobenzoic acid, diatrizoic acid, ioxitalamic acid, ioxaglic acid, iotroxic acid or any combination of them.

15. The X-ray contrast medium-albumin conjugate according to claim 14,
characterized in that
the X-ray contrast medium is a 2,3,5-triiodobenzoic acid.

16. The X-ray contrast medium-albumin conjugate according to claim 7,
characterized in that
the albumin is human serum albumin.

17. The X-ray contrast medium-albumin conjugate according to claim 7,
characterized in that
the albumin is present in its natural form.

18. The X-ray contrast medium-albumin conjugate according to claim 7,
characterized in that
the albumin is present in its native form.

19. The X-ray contrast medium-albumin conjugate according to claim 7,
characterized in that
the molar ratio of the X-ray contrast medium to albumin is 2:1 to 0.1:1.

20. A diagnostic or therapeutic agents comprising an X-ray contrast medium-albumin conjugate as the active substance which comprises a carboxyl group-containing X-ray contrast medium and albumin according to claim 7.

21. A process for producing an X-ray contrast medium-albumin conjugate, comprising reacting a carboxyl group-containing X-ray contrast medium with albumin in the presence of N-(3-dimethylaminopropyl)-N′-ethylcarbodiimide as the activation reagent without at least one of N-hydroxysuccinimide and N-hydroxysuccinamide, wherein the X-ray contrast medium is activated with the exclusion of water.

22. The process according to claim 22,
characterized in that
the carboxyl group-containing X-ray contrast medium is directly covalently coupled to albumin.

23. The process according to claim 22,
characterized in that
firstly an activated acid is formed from the carboxyl group-containing X-ray contrast medium using N-(3-dimethylaminopropyl)-N′-ethylcarbodiimide and subsequently the activated acid of the X-ray contrast medium is reacted with albumin.

24. The process according to claim 23,
characterized in that
the X-ray contrast medium and albumin are reacted in the presence of N-(3-dimethylaminopropyl)-N′-ethylcarbodiimide as the activation reagent without an additional activation reagent.

25. A process for producing an X-ray contrast medium-albumin conjugate,
characterized in that
an acid chloride of the X-ray contrast medium is produced from the carboxyl group-containing X-ray contrast medium using thionyl chloride or oxalyl chloride and said acid chloride is subsequently directly reacted with albumin.

26. The process according to claim 21,
characterized in that
the molar ratio of the X-ray contrast medium to albumin is 2:1 to 0.1:1.

27. The X-ray contrast medium-albumin conjugate according to claim 8,
obtainable by
directly covalently coupling the X-ray contrast medium to albumin.

28. The X-ray contrast medium-albumin conjugate according to claim 27,
obtainable by
coupling the X-ray contrast medium to albumin via an acid amide bond.

29. The X-ray contrast medium-albumin conjugate according to claim 8,
characterized in that
the X-ray contrast medium is an iodine compound.

30. The X-ray contrast medium-albumin conjugate according to claim 8,
characterized in that
the X-ray contrast medium is a triiodobenzoic acid.

31. The X-ray contrast medium-albumin conjugate according to claim 30,
characterized in that
the triiodobenzoic acid is a 2,3,5-triiodobenzoic acid, diatrizoic acid, ioxitalamic acid, ioxaglic acid and/or iotroxic acid.

32. The X-ray contrast medium-albumin conjugate according to claim 31,
characterized in that
the X-ray contrast medium is a 2,3,5-triiodobenzoic acid.

33. The X-ray contrast medium-albumin conjugate according to claim 8,
characterized in that
the albumin is human serum albumin.

34. The X-ray contrast medium-albumin conjugate according to claim 8,
characterized in that
the albumin is present in its natural form.

35. The X-ray contrast medium-albumin conjugate according to claim 8,
characterized in that
the albumin is present in its native form.

36. The X-ray contrast medium-albumin conjugate according to claim 8,
characterized in that
the molar ratio of the X-ray contrast medium to albumin is 2:1 to 0.1:1.

37. A diagnostic or therapeutic agent, comprising an X-ray contrast medium-albumin conjugate as the active substance which comprises a carboxyl group-containing X-ray contrast medium and albumin according to claim 8.

38. The process according to claim 25,
characterized in that
the molar ratio of the X-ray contrast medium to albumin is 2:1 to 0.1:1.
